(19) United States
(12) Patent Application Publication
(10) Pub. No.: US 2025/0171212 A1
(43) Pub. Date: May 29, 2025

(54) ZERO-EXPOSURE FORMALIN CONTAINER

(71) Applicant: Richard-Allan Scientific, LLC, Kalamazoo, MI (US)

(72) Inventors: Balazs Liposits, Budapest (HU); John Temple, Chester (GB)

(21) Appl. No.: 18/520,099

(22) Filed: Nov. 27, 2023

(51) Int. Cl. B65D 51/28; B01L 3/00; B65D 41/04

(52) U.S. Cl. CPC B65D 51/2835; B01L 3/502; B65D 41/04; B01L 2200/0689; B01L 2300/042; B01L 2300/044; B01L 2300/047; B01L 2300/0672

(57) ABSTRACT

Systems and methods of housing a medical specimen in a zero-exposure container are disclosed. In some embodiments, the zero-exposure container includes a container, a cap, and an insert. The cap may include a compartment for housing and sealing a liquid mixture, the compartment includes a film facing inwards relative to the system. The insert may include one or more protrusions operable to pierce the film of the compartment when the cap is fastened on the container while the system is in an active configuration. The insert may rest on an inner lip of the container such that the protrusions face downwards when the system is in a stored configuration and upwards when the system is in the active configuration. The liquid mixture may include a coloring agent that allows a user to visually verify the location of the liquid mixture in the system.

### BACKGROUND

### 1. Field

Embodiments of the present disclosure relate to medical containers. More specifically, embodiments of the present disclosure relate to zero-exposure, non-contact containers.

### 2. Related Art

Formaldehyde is well-known for being toxic to humans. For example, formaldehyde is thought to be a carcinogen. As such, it is often in the best interest of humans to limit their exposure to formaldehyde. Unfortunately, this can often be a difficult task, given that formaldehyde is a volatile organic compound (VOC), meaning it is emitted into the air, increasing the likelihood of human exposure.

Despite its many dangers, formaldehyde-containing products are widely used and are also beneficial. For example, formaldehyde is commonly found in resins, building materials, household products, fertilizers, pesticides, and preservatives. Formalin is a formaldehyde-containing product that is a well-known fixative used as part of the routine biopsy sample collection process, given that it is both economical and fast-acting. Therefore, given the great benefits of formalin to the medical field but also formaldehyde's danger to humans, it is desirable to limit human exposure to formaldehyde during the biopsy process. Thus, there is a need for systems that reduce human exposure to dangerous chemicals, including VOCs.

### SUMMARY

In some aspects, the techniques described herein relate to a container system for housing a specimen, including: a container for housing the specimen, the container including: a lower cavity configured to receive the specimen; and an inner rim including a rim surface; a cap configured to fasten to the container, including: a compartment housing a liquid mixture, the liquid mixture including a coloring agent; and a film sealing the liquid mixture in the compartment; and an insert sized to selectively rest on the rim surface in an active configuration, the insert including: a plurality of protrusions positioned to break the film when the cap is fastened to the container and the insert is in the active configuration; and a plurality of holes positioned to allow the liquid mixture to drain from the compartment to the lower cavity when the film is broken.

In some aspects, the techniques described herein relate to a container system, wherein the liquid mixture includes a volatile organic compound.

In some aspects, the techniques described herein relate to a container system, wherein the insert is sized to selectively rest on the rim surface in a stored configuration, and wherein the plurality of protrusions are positioned in the lower cavity when the insert is in the stored configuration.

In some aspects, the techniques described herein relate to a container system, wherein the cap is configured to be fastened to the container via cap threads corresponding to container threads.

In some aspects, the techniques described herein relate to a container system, wherein the insert further includes a column, wherein the cap further includes a divot configured to be inverted by the column when the cap is fastened to the container and the insert is in the active configuration.

In some aspects, the techniques described herein relate to a container system, wherein the compartment is ring-shaped, wherein the divot is centered in the cap such that the compartment surrounds the divot.

In some aspects, the techniques described herein relate to a container system, wherein the lower cavity houses a dilutant.

In some aspects, the techniques described herein relate to a container system for housing a specimen, including: a container for housing the specimen, including: a lower cavity configured to receive the specimen; and an inner rim, including a rim surface; a cap configured to fasten to the container, including: a compartment housing a liquid mixture; a film sealing the liquid mixture in the compartment; and an invertible divot indicative of a seal state of the film; and an insert sized to selectively rest on the rim surface in a stored configuration and in an active configuration, the insert including: a plurality of protrusions positioned in the lower cavity when the insert is in the stored configuration and to break the film when the cap is fastened to the container and the insert is in the active configuration; a column positioned in the lower cavity when the insert is in the stored configuration and to invert the invertible divot when the cap is fastened to the container and the insert is in the active configuration; and a plurality of holes positioned to allow the liquid mixture to drain from the compartment to the lower cavity when the film is broken.

In some aspects, the techniques described herein relate to a container system, wherein the liquid mixture includes a coloring agent.

In some aspects, the techniques described herein relate to a container system, wherein the liquid mixture includes formalin.

In some aspects, the techniques described herein relate to a container system, wherein the cap and the container form a sealed enclosure when the cap is fastened on the container.

In some aspects, the techniques described herein relate to a container system, wherein the lower cavity houses a dilutant.

In some aspects, the techniques described herein relate to a container system, wherein the liquid mixture includes a fixative for preserving a bodily tissue sample.

In some aspects, the techniques described herein relate to a container system, wherein the invertible divot is centered in the cap and the compartment surrounds the invertible divot.

In some aspects, the techniques described herein relate to a container system for housing a specimen, including: a container for housing the specimen, including: a lower cavity configured to receive the specimen; and an inner rim, including a rim surface; a cap configured to fasten to the container, including: a compartment housing a liquid mixture, the liquid mixture including a volatile organic compound and a coloring agent; a film sealing the liquid mixture in the compartment; and an invertible divot indicative of a seal state of the film; and an insert sized to selectively rest on the rim surface in a stored configuration and in an active configuration, the insert including: one or more protrusions positioned in the lower cavity when the insert is in the stored configuration and to break the film when the cap is fastened to the container and the insert is in the active configuration; a column positioned in the lower cavity when the insert is in the stored configuration and to invert the invertible divot when the cap is fastened to the container and the insert is in the active configuration; and one or more holes positioned to allow the liquid mixture to drain from the compartment to the lower cavity when the film is broken.

In some aspects, the techniques described herein relate to a container system, wherein the coloring agent includes eosin.

In some aspects, the techniques described herein relate to a container system, wherein the invertible divot changes color when inverted.

In some aspects, the techniques described herein relate to a container system, wherein the volatile organic compound is formaldehyde.

In some aspects, the techniques described herein relate to a container system, wherein the cap and the container form a liquid-tight system.

In some aspects, the techniques described herein relate to a container system, wherein the cap and the container form a gas-tight system.

This summary is provided to introduce a selection of concepts in a simplified form that are further described below in the detailed description. This summary is not intended to identify key features or essential features of the claimed subject matter, nor is it intended to be used to limit the scope of the claimed subject matter. Other aspects and advantages of the present disclosure will be apparent from the following detailed description of the embodiments and the accompanying drawing figures.

### BRIEF DESCRIPTION OF THE DRAWING FIGURES

Embodiments of the present disclosure are described in detail below with reference to the attached drawing figures, wherein:

FIG. 1 illustrates an exploded view of an exemplary zero-exposure container system, in accordance with embodiments of the invention;

FIGS. 2A-2C illustrates an exemplary zero-exposure container system, in accordance with embodiments of the invention;

FIG. 3 illustrates an exemplary exploded view of a zero-exposure container system, in accordance with embodiments of the invention;

FIGS. 4A-4C illustrate an exemplary zero-exposure container system, in accordance with embodiments of the invention; and

FIG. 5 illustrates an exemplary flowchart for illustrating the operation of a method, in accordance with embodiments of the invention.

The drawing figures do not limit the present disclosure to the specific embodiments disclosed and described herein. The drawings are not necessarily to scale, emphasis instead being placed upon clearly illustrating the principles of the present disclosure.

### DETAILED DESCRIPTION

The following detailed description references the accompanying drawings that illustrate specific embodiments in which the present disclosure can be practiced. The embodiments are intended to describe aspects of the present disclosure in sufficient detail to enable those skilled in the art to practice the present disclosure. Other embodiments can be utilized, and changes can be made without departing from the scope of the present disclosure. The following detailed description is, therefore, not to be taken in a limiting sense. The scope of the present disclosure is defined only by the appended claims, along with the full scope of equivalents to which such claims are entitled.

In this description, references to “one embodiment,” “an embodiment,” or “embodiments” mean that the feature or features being referred to are included in at least one embodiment of the technology. Separate references to “one embodiment,” “an embodiment,” or “embodiments” in this description do not necessarily refer to the same embodiment and are also not mutually exclusive unless so stated and/or except as will be readily apparent to those skilled in the art from the description. For example, a feature, structure, act, etc., described in one embodiment may also be included in other embodiments, but is not necessarily included. Thus, the technology can include a variety of combinations and/or integrations of the embodiments described herein.

In general, embodiments of the invention relate to a zero-exposure container system. The system may include a container, a cap, and an insert. The cap may include a compartment for housing and sealing a liquid mixture (such as a hazardous liquid mixture), the compartment including a film facing inwards relative to the system. The insert may include one or more holes and one or more protrusions designed to pierce the film of the compartment when the cap is fastened on the container while the system is in an active configuration. The insert may rest on an inner lip of the container such that the protrusions of the insert face downwards when the system is in a stored configuration and upwards when the system is in the active configuration. The container may receive a specimen such that when the system is in the active configuration, the liquid mixture mixes with the specimen and can perform its function, such as fixation. The liquid mixture may include a coloring agent that allows a user to visually verify the location of the liquid mixture in the system. In some embodiments, the system further includes a column attached to the insert and a divot in the cap such that the column inverts the divot when the protrusions have pierced the film of the compartment.

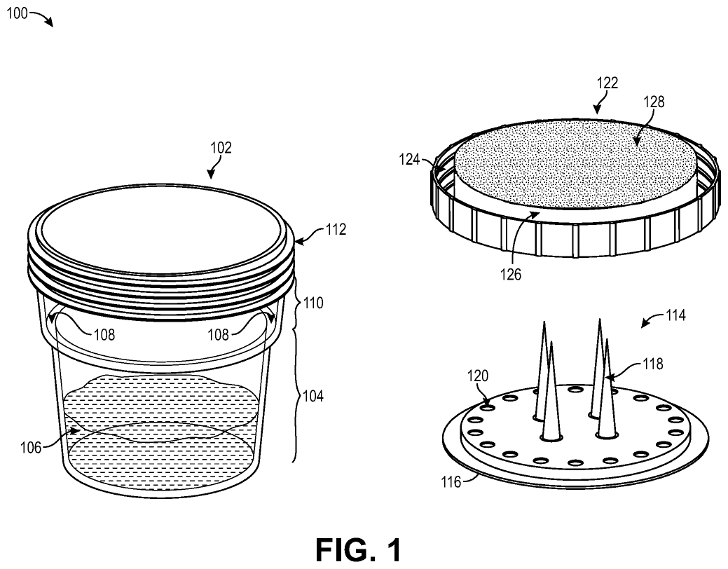

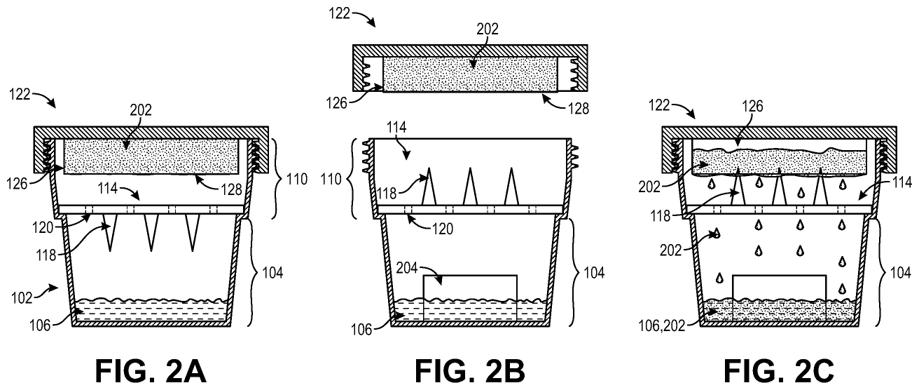

To begin, FIG. 1 depicts an exemplary zero-exposure container system in accordance with embodiments of the invention and generally referred to by reference numeral 100. In some embodiments, system 100 may include container 102, cap 122, and insert 114. System 100 may be scalable to any size. As discussed below with regard to FIGS. 2A-2C, system 100 may transition between a plurality of configurations, including a stored configuration and an active configuration.

Generally, system 100 may be a rigid or semi-rigid structure. The components of system 100 may be formed from any suitable material now known or later developed, including, but not limited to, plastic, silicon, glass, rubber, metal, cardboard, and other similar materials. In some embodiments, some or all components of system 100 may be formed from a transparent or semi-transparent material. System 100 and/or any components of system 100 may be any color or tint now known or later developed, including orange, pink, and amber. As described below, some or all the components of system 100 may be formed from materials that allow a user to see a color indicator to verify the location of a liquid mixture.

At a high level, system 100 includes container 102 for housing a specimen and a plurality of compounds. In some embodiments, container 102 may include a lower cavity 104. Lower cavity 104 may be configured to house a sample and/or additional substances. For example, lower cavity 104 may optionally house a dilutant 106. Examples of dilutant 106 may include water, alcohol, alcohol-based compounds, and similar substances. Dilutant 106 may be selected to dilute liquid mixture 202, depicted in FIGS. 2A-2C, such that all or a substantial portion of specimen 204, depicted in FIGS. 2B-2C, is covered by the mixture of dilutant 106 and liquid mixture 202. Lower cavity 104 may also receive a specimen such as specimen 204 depicted in FIG. 2B. In some embodiments, the specimen is a biopsy sample, such as bodily tissue. By placing a biopsy sample in the lower cavity 104, the sample may be safely preserved for further testing at a later date.

In some embodiments, container 102 may include inner rim 108 disposed above lower cavity 104. Inner rim 108 may form a ring around the inner diameter of container 102 and create a flat, or near flat, surface that is parallel, or near parallel, to the bottom base of container 102. The surface of inner rim 108 may receive insert 114 such that the outer edge of insert 114 rests flush on the surface of inner rim 108. The diameter of inner lip may be larger than lower cavity 104 such that base 116 of insert 114 may rest on the surface of inner rim 108 without falling into lower cavity 104. Accordingly, inner rim 108 may be a diameter that is smaller than that of the upper cavity 110. Further, inner rim 108 may have a diameter that is smaller than upper cavity 110 so that insert 114 may be removed from container 102 through upper cavity 110.

In some embodiments, container 102 may include an upper cavity 110 disposed above inner rim 108. Upper cavity 110 may house compartment 126 and/or protrusions 118 of insert 114, depending on the configuration of system 100. For example, when system 100 is in the stored configuration (as discussed below with regard to FIG. 2A), upper cavity 110 may receive (sealed) compartment 126 of cap 122 and the bottom face (i.e., the face opposite the face in which protrusions 118 are attached) of base 116 of insert 114. For another example, when system 100 is in the active configuration (as discussed below with regard to FIG. 2C), upper cavity 110 may receive protrusions 118 and compartment 126 of cap 122 so that protrusions 118 come into contact with film 128 of compartment 126. The various configurations are described in more detail below and illustrated in FIGS. 2A-2C.

In some embodiments, container 102 may include container threading 112 disposed about upper cavity 110, and cap 122 may include cap threading 124. Container threading 112 may be operable to receive cap threading 124 of cap 122, to fasten cap 122 onto container 102 and form a sealed enclosure. In some embodiments, the combination of cap 122 and container 102 forms a liquid-tight enclosure. In other embodiments the combination of cap 122 and container 102 forms a gas-tight enclosure. As such, the sealed enclosure may allow for the mixing of volatile organic compounds (or any other hazardous material) with a specimen without exposing the external environment to the volatile organic compound. It is noted herein that any and all securing methods are contemplated for securing cap 122 to container 102, including, but not limited to, snapping, clicking, latching, hinging, and other fastening methods.

At a high level, cap 122 may include compartment 126 for storing and sealing a substance or mixture. As discussed with respect to FIGS. 2A-2C, compartment 126 may house liquid mixture 202 to be combined with specimen 204 and dilutant 106 located in lower cavity 104. The compartment 126 may be formed from the inner side of cap 122, one or more walls protruding from the inner side of cap 122, and a film 128. In some embodiments, cap 122 may include a plurality of separated compartments, each housing a separate liquid mixture (for example, liquid mixtures that should not be mixed prior to being applied to the specimen). In such embodiments, a corresponding number of protrusions may be provided such that the film covering each compartment is pierced.

Film 128 may be opposite the inner side of cap 122 such that it is housed within upper cavity 110 as shown in FIGS. 2A-2C. Film 128 may be broken such that the contents of compartment 126 drain from compartment 126 into container 102, as depicted in FIG. 2C. For example, film 128 may be capable of being pierced by protrusions 118 of the insert 114 thereby forming one or more holes in film 128, allowing liquid mixture 202 to drip into lower cavity 104 of container 102. Thus, film 128 may be of the material thinner than the material forming the remainder of cap 122 and may be any material now known or later developed that allows for film 128 to be pierced, including, but not limited to, plastic, paper, and the like.

In some embodiments, compartment 126 may be sealed when system 100 is not in the active configuration, such as when in the stored configuration or transitional configuration described in FIGS. 2A-2C. For example, film 128 of compartment 126 may be unbroken when system 100 is not in the active configuration. As such, compartment 126 may prevent human exposure to liquid mixture 202 before a sealed enclosure is formed by fastening cap 122 on container 102.

As discussed above, system 100 may include insert 114. In some embodiments, insert 114 may include base 116, one or more of protrusions 118, and one or more of holes 120. Insert 114 may be formed from any suitable material now known or later developed, including, but not limited to, plastic, glass, silicon, rubber, and similar materials.

In some embodiments, insert 114 may include base 116. Base 116 includes a top face to which protrusions 118 are attached and a bottom face opposite the top face. Base 116 may be a rigid or semi-rigid structure such that when the bottom face of base 116 is resting directly on inner rim 108, a downward force may be placed on the top face of base 116 without causing insert 114 to cave towards lower cavity 104.

In some embodiments, base 116 may be circular in shape. As such, the outer diameter of base 116 may be sized to rest flush on inner rim 108. It is contemplated herein that base 116 may be any other shape suitable for resting on a portion or the entirety of inner rim 108. For example, base 116 may be rectangular, trapezoidal, and the like. In some embodiments, holes 120 are formed by virtue of base 116 being a different shape than inner rim 108. For example, base 116 may be square with diagonals equal to the diameter of a circular instance of inner rim 108, thereby allowing liquid mixture 202 to flow between the outer perimeter of the square and the inner circumference of the circle.

In some embodiments, as mentioned above, the top face of base 116 may include protrusions 118. Protrusions 118 may be any rigid or semi-rigid material that can pierce through film 128 of cap 122. For example, protrusions 118 may include sharp edges or end in a sharp point. As such, protrusions 118 may break a portion or all of film 128 such that liquid mixture 202 drains out of compartment 126 through one or more holes in film 128.

In some embodiments, base 116 may contain a lip or other handle. The lip may assist a user in grasping insert 114 so as to remove and/or flip over insert 114 when transitioning system 100 from the stored configuration to the active configuration (and vice versa). For example, to transition from the stored configuration to the active configuration, a user may grasp insert 114 from a resting position on inner rim 108, flip over insert 114 such that the bottom face of base 116 is facing downward, and replace insert 114 on inner rim 108. The lip may protrude from either the bottom face or the top face of base 116. In some embodiments, the lip may be part of protrusions 118 such that the lip may break the film 128.

In some embodiments, insert 114 may include one or more holes of 120. Holes 120 may allow for liquid mixture 202 to be transferred from compartment 126 in upper cavity 110 to lower cavity 104 (and vice versa). For example, upon the piercing of film 128 of compartment 126, liquid mixture 202 may drip down to insert 114 and drain through holes 120 into lower cavity 104. Holes 120 may be any shape necessary to allow for the drainage of liquid mixture 202. In some embodiments, holes 120 may be of a shape or size to allow for liquid to transfer between lower cavity 104 and upper cavity 110 at a predetermined rate.

FIGS. 2A-2C depict the transition of system 100 from the stored configuration to the active configuration. FIG. 2A depicts system 100 in the stored configuration. In some embodiments, when in the stored configuration, cap 122 may rest on top of container 102, may be fastened to the top of container 102, or may be separate from the top of container 102. Compartment 126 of cap 122, when in the stored configuration, may be sealed. Accordingly, compartment 126 may contain liquid mixture 202 in such a way as to prevent the exposure of liquid mixture 202 to the external environment. In some embodiments, when in the stored configuration, liquid mixture 202 may be tinted such that the color indicates the location of the liquid mixture 202. As such, a user can visually determine, for example, that system 100 is safe to handle because liquid mixture 202 is sealed in compartment 126 of cap 122.

Liquid mixture 202 stored in compartment 126 may be a hazardous material, such as formaldehyde, a formaldehyde compound, formalin, or other VOCs. In some embodiments, the hazardous material may include a chemical substance used to preserve biological material for later processing (i.e., a fixative). It is noted herein that liquid mixture 202 may be any substance and is not limited to hazardous materials.

Liquid mixture 202 may additionally include a coloring agent that tints liquid mixture 202 and/or changes the color of liquid mixture 202 to, for example, the color pink. Thus, the coloring agent can be used to determine, for example, that the formalin in liquid mixture 202 is properly sealed in compartment 126. Alternatively, the coloring agent may indicate that the formalin has drained into lower cavity 104 of container 102, thereby acting as a visual indicator that liquid mixture 202 has been successfully mixed with the specimen. Doing so may prevent specimen sample loss, as it provides a visual indicator that the specimen has been properly fixated after cap 122 is fastened on container 102.

The coloring agent may be any suitable substance for causing the color of the liquid mixture to change, such as a dye. In some embodiments, the coloring agent may provide additional functionality beyond color indication. For example, the coloring agent may contribute to the fixation process. For another example, the coloring agent may highlight one or more properties of the specimen. The coloring agent may be a fluorescent dye such as eosin, which may tint liquid mixture 202 with color while also allowing for tracking of small biopsy samples through tissue processing.

When in the stored configuration, insert 114 may be stored in container 102. In such embodiments, the top face of base 116, including protrusions 118, may directly contact the top surface of inner rim 108 such that protrusions 118 are pointed downwards towards lower cavity 104. Accordingly, protrusions 118 will not come into contact with film 128 of cap 122 in the event that cap 122 is fastened to container 102, thus preventing compartment 126 of cap 122 from becoming unsealed. In some embodiments, when in the stored configuration, insert 114 may be separate from container 102. For example, insert 114 may be stored separately from container 102 such that insert 114 need not be removed before receiving specimen 204, as described below.

In some embodiments, system 100 may be unable to receive specimen 204 while in the stored configuration. As such, system 100 may need to be transitioned to the active configuration in order to receive specimen 204. FIG. 2B depicts system 100 as it transitions from the stored configuration to the active configuration (i.e., the transitional configuration).

To transition from the stored configuration to the active configuration, cap 122 may be unfastened and removed from container 102 such that a user can remove insert 114. Next, insert 114 may be removed such that specimen 204 may be added to lower cavity 104 of container 102. It is an advantage of some embodiments that, when in the transitional configuration, compartment 126 of cap 122 remains sealed. Accordingly, the user and the external environment are not exposed to the liquid mixture 202 within compartment 126. Further, the user may be able to visually verify the sealed nature of compartment 126 in embodiments where a coloring agent is added to liquid mixture 202.

After the placement of specimen 204 in the lower cavity 104 of container 102, system 100 may then be transitioned to the active configuration by screwing cap 122 onto container 102 such that a sealed enclosure is formed. FIG. 2C depicts system 100 when in the active configuration. In the active configuration, no substances within system 100 are exposed to the environment outside system 100. For example, when system 100 is in the active configuration, any VOCs in liquid mixture 202 inside system 100 may not spread into the air outside.

In some embodiments, when transitioned to the active configuration, protrusions 118 of insert 114 come into contact with and pierce through film 128, thereby unsealing compartment 126 of cap 122. This may allow liquid mixture 202 in compartment 126 to drain into lower cavity 104. As such, the liquid mixture 202 may mix with the dilutant 106 and the specimen 204. In embodiments where liquid mixture 202 contains a coloring agent, this will change the appearance of dilutant 106. Thus, for example, if liquid mixture 202 is a fixative, a user can ensure the fixative is mixed with specimen 204 such that specimen 204 is preserved for later testing.

Overall, transitioning the system from the stored configuration to active configuration may transition compartment 126 from being sealed to being unsealed such that it minimizes external exposure by a user to the liquid mixture 202 in system 100 while also allowing the user to visually verify when the liquid mixture 202 has entered the sealed enclosure created by the container 102 and cap 122. Accordingly, both user exposure and sample loss may be prevented.

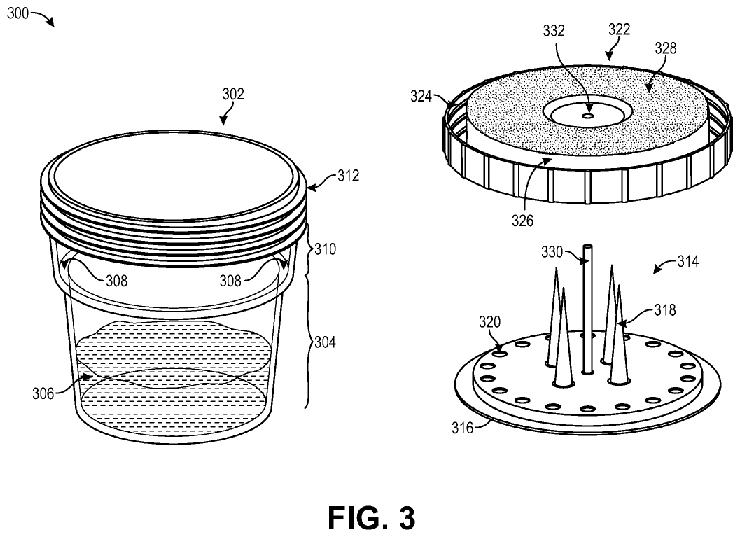

FIG. 3 depicts an alternative embodiment of a zero-exposure container, generally referred to by reference numeral 300. In some embodiments, system 300 may include container 302, generally corresponding to container 102 depicted in FIG. 1. Container 302 may include lower cavity 304, inner rim 308, and upper cavity 310, generally corresponding to lower cavity 104, inner rim 108, and upper cavity 110 depicted in FIG. 1, respectively. Lower cavity 304 may contain dilutant 306, generally corresponding to dilutant 106 depicted in FIG. 1. A container threading 312, generally corresponding to container threading 112 depicted in FIG. 1, may be disposed above upper cavity 310 for receiving a cap 322.

In some embodiments, cap 322 may include cap threading 324, corresponding generally to cap threading 124 depicted in FIG. 1. Cap threading 324 may be operable to fasten to container threading 312 of container 302. Cap 322 may include a compartment 326 attached to the underside of cap 322. Compartment 326 may be sealed by a film 328, film 328 being attached to compartment 326 such that film 328 faces towards upper cavity 310.

In some embodiments, compartment 326 is ring-shaped and/or donut-shaped to accommodate a divot 332 located approximately in the center of cap 322. For example, compartment 326 may be ring-shaped such that it surrounds divot 332. However, it is contemplated herein that divot 332 need not be in the center of cap 322 and can instead be located anywhere on cap 322. Broadly, divot 332 may indicate when one or more of protrusions 318 (generally corresponding to protrusions 118 depicted in FIG. 1) have pierced film 328 of compartment 326. Divot 332 may be configured to invert from a concave configuration relative to upper cavity 310 to a convex configuration when pressure is applied upward relative to container 302. For example, divot 332 may be concave towards the inside of container 302 while in a stored configuration and a transitional configuration. Accordingly, divot 332 may invert outwards when system 300 is in an active configuration, and protrusions 318 of insert 314 have pierced film 328 of compartment 326. As such, divot 332 may be formed from a material such that it may invert from a concave configuration to a convex configuration when pressure is applied to divot 332. In some embodiments, divot 332 changes color when inverted. For example, mechanical stress on divot 332 may cause a change in the color of divot 332. Alternatively, divot 332 may be transparent such that, once inverted a color of the tip of column 330 is visible once it has inverted divot 332.

In some embodiments, divot 332 may be inverted outwards relative to cap 322 by column 330. Generally, column 330 may be attached to the top face of insert 314. Column 330, at a high level, may provide a visual indication to a user that protrusions 318 of insert 314 have pierced compartment 326 of cap 322. Column 330 may be formed from a rigid or semi-rigid material and may be formed from any suitable material, including plastic, glass, silicone, rubber, or metal. Column 330 may be any suitable shape, such as cylindrical, rectangular, or prismatic.

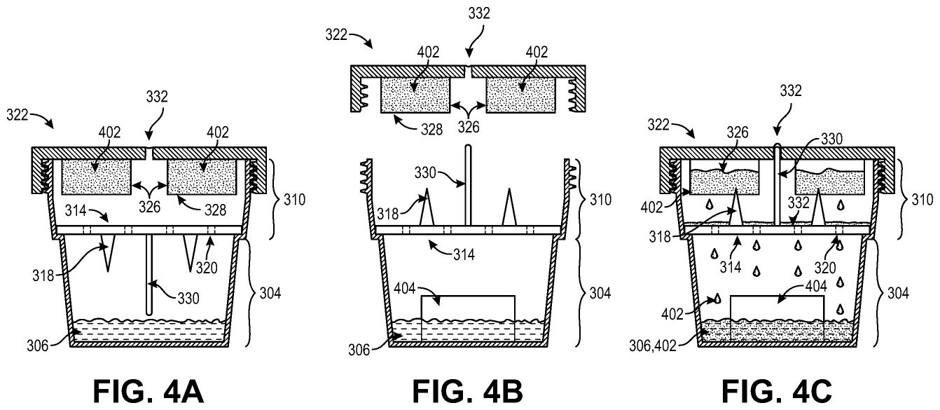

Column 330 may be in addition to or an alternative for the coloring agent of liquid mixture 402 discussed below with respect to FIGS. 4A-4C. Any number of additional indicators may be present in system 300 (or system 100) to alert the user that compartment 326 has been unsealed. For example, an additional component of container 302 or cap 322 may be configured to audibly click to indicate when cap 322 is fastened on container 302 such that film 328 of compartment 326 is broken.

In some embodiments, column 330 is attached to the top face of base 316 (generally corresponding to base 116 depicted in FIG. 1) of insert 314 in a similar fashion to protrusions 318. When in the stored configuration, column 330 may face downward such that it protrudes into lower cavity 304 of container 302. When in an active configuration, column 330 may face upward such that it rests in the upper cavity 310 of container 302 and protrudes through cap 322 to invert divot 332 of cap 322.

FIGS. 4A-4C depict the transition of system 300 from the stored configuration to the active configuration. FIG. 4A depicts system 300 in the stored configuration. The stored configuration of system 300 generally corresponds to the stored position of system 100. In some embodiments, when in the stored configuration, cap 322 may be fastened to container 302 such that compartment 326 faces downwards into upper cavity 310. In the stored configuration, insert 314 may be oriented such that the top face of base 316 rests on inner rim 308. Thus, in the stored configuration, inner rim 308 and column 330 protrude downwards into lower cavity 304.

In some embodiments, when in the stored configuration, liquid mixture 402, generally corresponding to liquid mixture 202 depicted in FIG. 2A, may be sealed in compartment 326. As such, a user of system 300 may visually verify that liquid mixture 402 is sealed and has not been mixed with dilutant 306 and specimen 404 (generally corresponding to specimen 204 depicted in FIG. 2B). The user may further verify liquid mixture 402 is sealed within compartment 326 by observing the orientation of divot 332. For example, if a user observes that divot 332 is orientated such that it is concaved inward relative to cap 322, the user may verify protrusions 318 have not broken film 328 and compartment 326 remains sealed.

FIG. 4B depicts system 300 in a transitional configuration in accordance with embodiments of the invention. Generally, the transitional configuration of system 300 is in line with the transitional configuration of system 100 depicted in FIG. 2B. In some embodiments, when in the transitional configuration, insert 314 is removed, specimen 404 is placed in lower cavity 304, and insert 314 is returned to container 302.

When transitioning between the stored configuration and the active configuration, a user may change the orientation of insert 314 such that protrusions 318 and column 330 face upwards into upper cavity 310. As such, the user may also remove cap 322 from container 302 to ensure compartment 326 remains sealed, and liquid mixture 402 is not exposed to the external environment. To verify cap 322 has remained sealed while specimen 404 has been placed in lower cavity 304, the user may observe the location of liquid mixture 402 as well as the orientation of divot 332, as described above with respect to FIG. 4A.

FIG. 4C depicts system 300 in the active configuration in accordance with embodiments of the invention. Generally, the active configuration of system 300 is in line with the active configuration of system 100 depicted in FIG. 2C. In some embodiments, when in the active configuration, cap 322 may be fastened onto container 302 such that protrusions 318 tear and/or break film 328 of compartment 326. Accordingly, liquid mixture 402 may drain from compartment 326 and flow through one or more of holes 320 into lower cavity 304. When in lower cavity 304, liquid mixture 402 may mix with specimen 404 and dilutant 306.

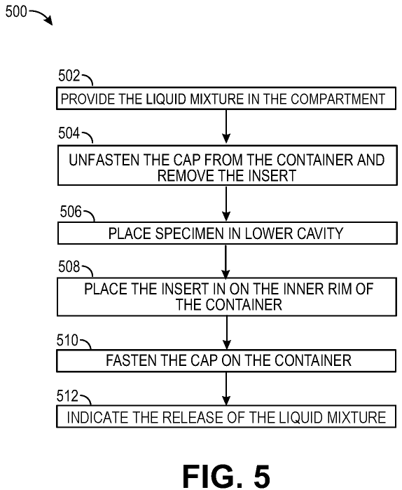

Continuing on, FIG. 5 depicts an exemplary method for using a zero-exposure container system in accordance with embodiments of the invention and generally referred to by reference numeral 500. Method 500 may relate to a method for utilizing system 100 and/or system 300 for preserving a biopsy sample for future testing and processing. Method 500 is generally described with reference to elements of system 300 but is equally applicable to system 100.

In step 502, liquid mixture 402 is provided in compartment 326 of cap 322. In some embodiments, the location of liquid mixture 402 may be visible due to the addition of a coloring agent in liquid mixture 402. Accordingly, the user may be able to visually determine where liquid mixture 402 is in the system (for example, in compartment 326 of cap 322) by observing the location of color in the system.

In step 504, cap 322 is unfastened from container 302 and insert 314 is removed. In some embodiments, cap 322 includes cap threading 324 such that cap 322 is removed by unscrewing. Prior to removal, insert 314 may be oriented such that protrusions 318 and column 330 are facing downwards into lower cavity 304 of container 302. As discussed above with respect to FIG. 2A, insert 314 may be removed by a user via the lip. In some embodiments, insert 314 may be separate from container 302 and, therefore, need not be removed from container 302.

In step 506, specimen 404 may be placed into dilutant 306 in lower cavity 304 of container 302. Specimen 404 may be any sample suitable for housing in system 300. For example, specimen 404 may be a bodily tissue sample recovered from a biopsy procedure. Accordingly, the bodily tissue may be placed in system 300 to preserve for future testing at a separate location from that in which the bodily tissue was extracted.

In step 508, insert 314 may be placed to rest on inner rim 308 of container 302 such that protrusions 318 and column 330 face upwards into upper cavity 310 of container 302. As such, insert 314 is oriented to break film 328 of compartment 326 (which houses liquid mixture 402) when cap 322 is fastened onto container 302.

In step 510, cap 322 is fastened to container 302. In some embodiments, upon fastening cap 322 to container 302, protrusions 318 pierce film 328 of compartment 326 such that liquid mixture 402 flows into the remaining cavity of container 302. Liquid mixture 402 may flow then through holes 320 of insert 314 into lower cavity 304. As such, the liquid mixture 402 may then mix with specimen 404 and any other base substances (such as dilutant 306) located in the enclosure of container 302 and cap 322. Liquid mixture 402 may have any number of functionalities with respect to specimen 404, including fixation. In some embodiments, the user may be able to observe the location of liquid mixture 402 to now be in lower cavity 304 due to a coloring agent added to liquid mixture 402.

In step 512, the release of liquid mixture 402 is indicated. In some embodiments, the orientation of divot 332 in the cap is changed to indicate the release of liquid mixture 402 from compartment 326. As discussed above, the orientation of divot 332 corresponds to whether film 328 of compartment 326 has been pierced and liquid mixture 402 has drained into the enclosure formed by container 302 and cap 322. For example, if divot 332 is concave inwards relative to the system, the user can know that liquid mixture 402 remains sealed in compartment 326. Alternatively, if divot 332 is convex outwards relative to the system, the user may verify that liquid mixture 402 has drained into the enclosure and has thus mixed with specimen 404. In some embodiments, divot 332 in cap 322 may be observed visually or by touch. For example, the user may visually observe divot 332 as protruding from cap 322 or run a finger across cap 322 and feel divot 332 protruding from cap 322.

In other embodiments, the color of dilutant 306 is changed to indicate the release of liquid mixture 402 from compartment 326. As discussed above, a coloring agent may be added to liquid mixture 402, allowing a user to determine the location of liquid mixture 402 in the system. When liquid mixture 402 mixes with specimen 404 and dilutant 306, dilutant 306 may also change color, allowing a user to determine the liquid mixture 402 has mixed with dilutant 306 and specimen 404. As such, the release of liquid mixture 402 from compartment 326 may be visually verified by observing color in dilutant 306 and/or specimen 404. Conversely, a user may visually verify that liquid mixture 402 remains sealed in compartment 326 by observing the location of colored liquid as being confined to compartment 326. In some embodiments, the admixture of the coloring agent and the dilutant may cause a chemical reaction between the coloring agent and the dilutant, such that a color change in the mixture gives a color different from the pre-mixture color of the coloring agent and the pre-mixture color of the dilutant.

Features described above as well as those claimed below may be combined in various ways without departing from the scope hereof. The following examples illustrate some possible, non-limiting combinations:

(A1) A container system for housing a specimen, comprising: a container for housing the specimen, the container comprising: a lower cavity configured to receive the specimen; and an inner rim including a rim surface; a cap configured to fasten to the container, comprising: a compartment housing a liquid mixture, the liquid mixture comprising a coloring agent; and a film sealing the liquid mixture in the compartment; and an insert sized to selectively rest on the rim surface in an active configuration, the insert comprising: a plurality of protrusions positioned to break the film when the cap is fastened to the container and the insert is in the active configuration; and a plurality of holes positioned to allow the liquid mixture to drain from the compartment to the lower cavity when the film is broken.

(A2) For the container system denoted as (A1), wherein the liquid mixture comprises a volatile organic compound.

(A3) For the container system denoted as (A1) or (A2), wherein the insert is sized to selectively rest on the rim surface in a stored configuration, and wherein the plurality of protrusions are positioned in the lower cavity when the insert is in the stored configuration.

(A4) For container system denoted as (A1) through (A3), wherein the cap is configured to be fastened to the container via cap threads corresponding to container threads.

(A5) For container system denoted as (A1) through (A4), wherein the insert further comprises a column, wherein the cap further comprises a divot configured to be inverted by the column when the cap is fastened to the container and the insert is in the active configuration.

(A6) For container system denoted as (A1) through (A5), wherein the compartment is ring-shaped, wherein the divot is centered in the cap such that the compartment surrounds the divot.

(A7) For container system denoted as (A1) through (A6), wherein the lower cavity houses a dilutant.

(B1) A container system for housing a specimen, comprising: a container for housing the specimen, comprising: a lower cavity configured to receive the specimen; and an inner rim, comprising a rim surface; a cap configured to fasten to the container, comprising: a compartment housing a liquid mixture; a film sealing the liquid mixture in the compartment; and an invertible divot indicative of a seal state of the film; and an insert sized to selectively rest on the rim surface in a stored configuration and in an active configuration, the insert comprising: a plurality of protrusions positioned in the lower cavity when the insert is in the stored configuration and to break the film when the cap is fastened to the container and the insert is in the active configuration; a column positioned in the lower cavity when the insert is in the stored configuration and to invert the invertible divot when the cap is fastened to the container and the insert is in the active configuration; and a plurality of holes positioned to allow the liquid mixture to drain from the compartment to the lower cavity when the film is broken.

(B2) For the container system denoted as (B1), wherein the liquid mixture comprises a coloring agent.

(B3) For the container system denoted as (B1) or (B2), wherein the liquid mixture comprises formalin.

(B4) For container system denoted as (B1) through (B3), wherein the cap and the container form a sealed enclosure when the cap is fastened on the container.

(B5) For container system denoted as (B1) through (B4), wherein the lower cavity houses a dilutant.

(B6) For container system denoted as (B1) through (B5), wherein the liquid mixture comprises a fixative for preserving a bodily tissue sample.

(B7) For container system denoted as (B1) through (B6), wherein the invertible divot is centered in the cap and the compartment surrounds the invertible divot.

(C1) A container system for housing a specimen, comprising: a container for housing the specimen, comprising: a lower cavity configured to receive the specimen; and an inner rim, comprising a rim surface; a cap configured to fasten to the container, comprising: a compartment housing a liquid mixture, the liquid mixture comprising a volatile organic compound and a coloring agent; a film sealing the liquid mixture in the compartment; and an invertible divot indicative of a seal state of the film; and an insert sized to selectively rest on the rim surface in a stored configuration and in an active configuration, the insert comprising: one or more protrusions positioned in the lower cavity when the insert is in the stored configuration and to break the film when the cap is fastened to the container and the insert is in the active configuration; a column positioned in the lower cavity when the insert is in the stored configuration and to invert the invertible divot when the cap is fastened to the container and the insert is in the active configuration; and one or more holes positioned to allow the liquid mixture to drain from the compartment to the lower cavity when the film is broken.

(C2) For the container system denoted as (C1), wherein the coloring agent comprises eosin.

(C3) For the container system denoted as (C1) or (C2), wherein the invertible divot changes color when inverted.

(C4) For container system denoted as (C1) through (C3), wherein the volatile organic compound is formaldehyde.

(C5) For container system denoted as (C1) through (C4), wherein the cap and the container form a liquid-tight system.

(C6) For container system denoted as (C1) through (C5), wherein the cap and the container form a gas-tight system.

Although the present disclosure has been described with reference to the embodiments illustrated in the attached drawing figures, it is noted that equivalents may be employed and substitutions made herein without departing from the scope of the present disclosure as recited in the claims.

Having thus described various embodiments of the present disclosure, what is claimed as new and desired to be protected by Letters Patent includes the following:

1. A container system for housing a specimen, comprising:
a container for housing the specimen, the container comprising:
a lower cavity configured to receive the specimen; and
an inner rim including a rim surface;
a cap configured to fasten to the container, comprising:
a compartment housing a liquid mixture, the liquid mixture comprising a coloring agent; and
a film sealing the liquid mixture in the compartment; and
an insert sized to selectively rest on the rim surface in an active configuration, the insert comprising:
a plurality of protrusions positioned to break the film when the cap is fastened to the container and the insert is in the active configuration; and
a plurality of holes positioned to allow the liquid mixture to drain from the compartment to the lower cavity when the film is broken.

2. The container system of claim 1, wherein the liquid mixture comprises a volatile organic compound.

3. The container system of claim 1,
wherein the insert is sized to selectively rest on the rim surface in a stored configuration,
wherein the plurality of protrusions are positioned in the lower cavity when the insert is in the stored configuration.

4. The container system of claim 1, wherein the cap is configured to be fastened to the container via cap threads corresponding to container threads.

5. The container system of claim 4,
wherein the insert further comprises a column,
wherein the cap further comprises a divot configured to be inverted by the column when the cap is fastened to the container and the insert is in the active configuration.

6. The container system of claim 5,
wherein the compartment is ring-shaped,
wherein the divot is centered in the cap such that the compartment surrounds the divot.

7. The container system of claim 1, wherein the lower cavity houses a dilutant.

8. A container system for housing a specimen, comprising:
a container for housing the specimen, comprising:
a lower cavity configured to receive the specimen; and
an inner rim, comprising a rim surface;
a cap configured to fasten to the container, comprising:
a compartment housing a liquid mixture;
a film sealing the liquid mixture in the compartment; and
an invertible divot indicative of a seal state of the film; and
an insert sized to selectively rest on the rim surface in an active configuration, the insert comprising:
a plurality of protrusions positioned to break the film when the cap is fastened to the container and the insert is in the active configuration;
a column positioned to invert the invertible divot when the cap is fastened to the container and the insert is in the active configuration; and
a plurality of holes positioned to allow the liquid mixture to drain from the compartment to the lower cavity when the film is broken.

9. The container system of claim 8, wherein the liquid mixture comprises a coloring agent.

10. The container system of claim 8, wherein the liquid mixture comprises formalin.

11. The container system of claim 8, wherein the cap and the container form a sealed enclosure when the cap is fastened on the container.

12. The container system of claim 8, wherein the lower cavity houses a dilutant.

13. The container system of claim 8, wherein the liquid mixture comprises a fixative for preserving a bodily tissue sample.

14. The container system of claim 8, wherein the invertible divot is centered in the cap and the compartment surrounds the invertible divot.

15. A container system for housing a specimen, comprising:
a container for housing the specimen, comprising:
a lower cavity configured to receive the specimen; and
an inner rim, comprising a rim surface;
a cap configured to fasten to the container, comprising:
a compartment housing a liquid mixture,
the liquid mixture comprising a volatile organic compound and a coloring agent;
a film sealing the liquid mixture in the compartment; and
an invertible divot indicative of a seal state of the film; and
an insert sized to selectively rest on the rim surface in an active configuration, the insert comprising:
one or more protrusions positioned to break the film when the cap is fastened to the container and the insert is in the active configuration;
a column positioned to invert the invertible divot when the cap is fastened to the container and the insert is in the active configuration; and
one or more holes positioned to allow the liquid mixture to drain from the compartment to the lower cavity when the film is broken.

16. The container system of claim 15, wherein the coloring agent comprises eosin.

17. The container system of claim 15, wherein the invertible divot changes color when inverted.

18. The container system of claim 15, wherein the volatile organic compound is formaldehyde.

19. The container system of claim 15, wherein the cap and the container form a liquid-tight system.

20. The container system of claim 19, wherein the cap and the container form a gas-tight system.
